(19) United States
(12) Patent Application Publication
(10) Pub. No.: US 2005/0096695 A1
(43) Pub. Date: May 5, 2005

(54) Flexible foam seal assembly

(76) Inventor: Jack M. Olich, Mahopac, NY (US)

Correspondence Address: LAWRENCE CRUZ, 12 GOOD HILL RD., BETHEL, CT, 06801, US

(21) Appl. No.: 10/698,109

(22) Filed: Nov. 3, 2003

(51) Int. Cl. A61D 1/00; A61B 17/08

(52) U.S. Cl. 606/213

(57) ABSTRACT

A seal for use in a cannula or tube, such as a surgical trocar cannula, is adapted to form a pressure barrier seal between two spaces and is adapted to accommodate a wide range of instrument diameters and objects passed therethrough. The present invention is directed to a generally cylindrical seal plug made of flexible foam materials and having a central orifice extending through along a central axis. In the preferred embodiments, the foam material is preferably a flexible foam material such as polyurethane foam.

### RELATED APPLICATIONS

None.

### TECHNICAL FIELD

The present invention relates to seal assemblies for pressure sealing between two volumes of differing pressure and, more particularly, for providing a sealed port through a pressure barrier that facilitates the passage therethrough of objects or instruments of varying size and shapes while substantially maintaining a pressure differential between two spaces.

### BACKGROUND AND OBJECTS OF THE INVENTION

There are a variety of applications in which it is desirable to maintain a pressure barrier between two adjacent spaces of relatively different pressures while facilitating the passage of objects or instruments of varying size and shape through the barrier with minimal pressure change or pressure loss. Such applications include, for example, biological or chemical laboratory systems having enclosed, pressurized chambers. Other applications include surgical procedures in which a body cavity is insufflated and objects or surgical instruments must be passed into and out of the body cavity. In such instances, it may be desirable to maintain a particular pressure differential between opposite sides of the pressure barrier to contain substances or contaminants and prevent them from passing through the pressure barrier, or to create and maintain the boundaries of a work space. It is desirable to achieve these objectives while facilitating the passage of varying sizes and shapes of objects or instruments through the pressure barrier.

Such devices are known in the context of laparoscopic access cannulas and trocar systems that are commonly used in minimally invasive surgical procedures. In particular types of laparoscopic procedures, a patient's abdominal cavity is inflated by piercing a hollow needle through the skin and subcutaneous muscle layers and introducing a pressurized gas into the cavity. The cavity is expanded to create a hollow space within. One or more trocar cannula assemblies including a bladed trocar knife nested concentrically within a cannula tube are inserted through the skin so that a distal end of the assembly is positioned within the cavity and a proximal end of the assembly remains outside the cavity, exposed to the ambient environment. Then the trocar blade is removed, leaving the cannula tube in place to serve as a port for inserting and removing instruments and objects.

The cannula tube has a septum seal with a central orifice and a “zero seal” positioned distally of the septum seal. The zero seal forms a first seal barrier and is typically in the form of a duckbill seal or a flapper seal. The septum seal is located proximally of the zero seal and is in the form of a stretched membrane across the diameter of the inside of the cannula tube. The septum seal has a central opening or orifice that is adapted to stretch in order to accommodate instruments or objects of greater diameter. The orifice forms a pressure seal around an instrument shaft when an instrument shaft is positioned therethrough. As the instrument is advanced distally, it pushes and holds open the zero seal, allowing pressurized gas to pass proximally past the zero seal. At this point, the pressurized gas is stopped at the septum seal since the instrument shaft is occupying the orifice and, thus, preventing gas from exiting through the orifice. When the instrument is removed from the zero seal it will re-close and resume its sealing function while the septum seal will lose its ability to seal when the instrument is removed completely from it.

A shortcoming of such trocar cannula systems as described above exists in the inability of a single orifice seal to effectively accommodate the use of a wide range of sizes of instrument shaft diameters positioned therethrough and irregularly shaped instrument heads that must pass through. A common deficiency in these types of systems is the tendency of the opening in an orifice seal to lack the resiliency to accommodate relatively large diameter instruments without tearing if it is sized small enough to accommodate relatively small diameter instruments. If the orifice is sized large enough to accommodate relatively large diameter instruments, it usually means that the orifice is sized too large to accommodate relatively small diameter instruments without experiencing a “cat-eying” problem. The orifice tends to cat-eye, or stretch, when a surgeon moves a relatively small diameter instrument laterally while it is positioned through the orifice. The orifice stretches out of round such that a portion loses contact with the outside diameter of the instrument shaft and results in pressurized gas escaping therethrough.

To overcome such shortcomings, multiple cannulas must be inserted into a patient so that several sizes of orifice seal are available. Another known solution is to provide a trocar cannula that has multiple seal templates or adapters that must be manipulated each time a different sized instrument is used. Yet another known solution is to use a floating septum seal that slides laterally relative to the cannula, wherein the seal floats between wiper contact sealing surfaces. Even yet another known solution is to provide a septum seal on a spherically movable mount such that the seal can move laterally relative to the cannula tube.

Each of the above-described solutions to the cat-eyeing problem have their own inherent shortcomings. For example, each adds complexity in construction, addition of costs for materials and manufacture, additional undesirable occupancy of space to accommodate additional structure, and cumbersome tactile feel or prohibition of movement experienced by physicians using such devices.

It is an object, therefore, to provide a simple, inexpensive and effective universal seal system for a cannula such as a surgical trocar cannula that avoids the shortcomings of the known devices described above.

These and other objects of the present invention are described herein or inherent to the present invention.

### SUMMARY OF THE INVENTION

The present invention is directed to a seal for use in a cannula or tube, such as a surgical trocar cannula, that is adapted to form a pressure barrier seal between two spaces and that is adapted to accommodate a wide range of instrument diameters and objects passed therethrough. While the preferred embodiments of the present invention are described with respect to laparoscopic surgery trocar systems by way of example, the scope of the present invention is not limited to laparoscopic or surgical applications, and the present invention has various applications.

The various preferred embodiments of the present invention described herein are directed to a generally cylindrical seal plug made of flexible foam materials and having a central orifice extending through along a central axis. In the preferred embodiments, the foam material is preferably a flexible foam material such as polyurethane foam, although in alternative situations, as described below, other foam material may be used.

Material properties related to elasticity of foams, both closed-cell and open-cell, are not possible to determine with precision, largely due to the non-uniformity of size, shape, and arrangement of individual cells in a given volumetric sample of most foams. Thus, specific parameters related to material characteristics, dimensions, and intended use conditions in combination with experimentation have led to the discovery of the preferred embodiments of the present invention herein described.

In a first preferred embodiment of the present invention, a cylindrical plug is made from a flexible cell foam. The plug has a central orifice therethrough that is sized slightly smaller than the smallest diameter of an instrument that will be positioned therein. The plug is sized with an outside diameter that is slightly smaller than the inside diameter of its surrounding housing structure. Similarly, the axial length of the plug is slightly less than the axial length of the space within which it is contained. Because of its sizing, the foam plug is designed to accommodate a variety of instrument shaft diameters. The foam plug will compress and displace according to the diameter of instrument placed therethrough and subsequent manipulation of the instrument.

In another embodiment of the preferred invention, the outside diameter of the foam plug, or the axial height, or both, is greater than the inside dimension of the cylindrical housing within which it is positioned. This allows a pre-stressed condition to exist, placing the plug in a compressed state in the radial direction, axial direction, or both, when no instrument is placed therein. The effect is that the central orifice is contracted in size prior to instrument insertion and the entire plug is always under compression during use, thereby ensuring that the plug material will always close a gap under conditions that normally cause cat-eyeing in conventional designs. Depending on the amount of radial force and other parameters such as the shape of the orifice, this may decrease the diameter of the orifice to the extent it is maintained in a closed position when no instrument is inserted therethrough.

In another variation of this embodiment there are no ceiling or floor constraints, enabling the plug to deform in the axial direction when an object placed in the orifice increases the radial force exerted on the inside wall of the cannula.

In one variation of this embodiment, the plug can be anchored against lateral movement. In another variation of this embodiment, the plug is not anchored so that it may slide laterally if an instrument positioned within the orifice is moved laterally.

A foam plug seal according to the present invention may be used with a variety of outside housing and mounting configurations such as, for example, a housing that has a first cover with a first sized central opening and a flip-top lid for selectively converting the first sized central opening to a second sized opening; or a housing to which the seal is fixedly mounted wherein the housing pivots along a spherical surface in a universal or three-dimensional sense.

### BRIEF DESCRIPTION OF THE DRAWINGS

FIG. 1 is a schematic, side cross-sectional view of a first embodiment of the present invention.

FIGS. 2A-2D are perspective views of a foam seal component of the present invention.

FIG. 2E is a schematic, cross-sectional view of a trocar housing according to the present invention.

FIG. 3A is an exploded view of a seal assembly according to the present invention.

FIG. 3B is an assembled view of a seal assembly according to FIG. 3A.

FIG. 4A is an exploded view of a seal assembly according to another embodiment of the present invention.

FIG. 4B is an assembled view of a seal assembly according to FIG. 4A.

FIG. 5 is a cross-sectional view of a foam seal according to another embodiment of the present invention.

FIG. 6 is a cross-sectional view of a foam seal according to another embodiment of the present invention.

FIG. 7 is a perspective view of a foam seal according to another embodiment of the present invention.

FIG. 8 is a perspective view of a foam seal according to another embodiment of the present invention.

FIG. 9 is a perspective view of a foam seal according to another embodiment of the present invention.

FIG. 10A is a schematic, top view of a seal assembly according to another embodiment of the present invention.

FIG. 10B is a schematic, side cross-sectional view of a seal assembly according to FIG. 10A.

FIG. 11A is a schematic, side cross-sectional view of a seal assembly according to another embodiment of the present invention.

FIG. 11B is a schematic, side cross-sectional view of a seal assembly according to FIG. 11A and having an instrument inserted therein.

FIGS. 12A-12B are top views of a seal component according to another embodiment of the present invention.

FIG. 13 is a schematic, cross-sectional view of a seal component according to another embodiment of the present invention.

FIG. 14 is a schematic, cross-sectional view of a seal assembly according to another embodiment of the present invention.

FIG. 15 is a schematic, perspective view of a seal assembly according to another embodiment of the present invention.

### DESCRIPTION OF THE PREFERRED EMBODIMENTS

The intended use parameters, including the range of instrument and object sizes and shapes to be inserted through the plug, as well as the pressure loads on either side of the plug during use, will determine the selection of foam cell size and material composition, plug dimensions, and the specific type and amount of pre-stress within the embodiments described herein. By way of example, the preferred embodiments disclosed herein are directed to a surgical trocar application in which instrument shafts intended to be passed through the plug range from about 4 millimeter (0.157 inches) to about 15 millimeters (0.591 inches). The inside diameter of the cannula tube is about 12.4 millimeters (0.49 inches). The pressures on each side of the plug range from pressures experienced at ambient temperature and pressure conditions to pressures ordinarily encountered during surgical laparoscopic procedures in which the abdominal cavity is insufflated.

The material selected for use in the plug in the preferred embodiments described herein is a flexible foam comprising cells that are about 0.32 millimeters (0.125 inches) in diameter. The material composition of the foam is polyurethane. This material is commercially available from Dynamic Systems Inc. and is known as Pudgee™. It is understood that this is one example of different flexible foam materials that may be implemented within the scope of the present invention.

Referring to FIG. 1, in an un-stressed state as would exist prior to insertion of an instrument, a foam plug (10) according to the present invention has a central orifice (12), an outer circumferential surface (14), an outer diameter (16), and an inner diameter (18). The plug (10) is housed in a generally cylindrical housing (20) having a top surface (22), a bottom surface (24), and a cylindrical side wall (26). The housing (20) has a top central orifice (28) and a lower central orifice (30) that opens into an elongated cannula (32).

Referring to FIG. 2E, the housing (20) has an outer diameter of Xh, and a height of Yh. Now, with reference to the various embodiments shown in FIGS. 2A-2D, each embodiment of a plug according to the present invention is described with reference to being housed in the housing (20) of FIG. 2E. For the description of FIGS. 2A-2E, the reference symbols X, Y, Xh, Yh, X1, and Y1 are related as: X<Xh<X1 and Y<Yh<Y1.

First, referring to FIG. 2A, a plug (34) of the type described in FIG. 1 has an outside diameter X and a vertical height Y. Because X and Y are less than Xh and Yh, respectively, there exists no pre-stressed condition when the plug (34) is positioned in the housing (20).

As shown in FIG. 2B, a plug (36) of the type described in FIG. 1 has an outside diameter X1 and a vertical height Y. Because X1 is greater than Xh and Y is less than Yh, there exists no pre-stressed condition with respect to the vertical or axial direction when the plug (36) is positioned in the housing (20); but there does exist a pre-stressed compression condition with respect to the radial direction along the direction of X1. This pre-stressed condition causes the orifice (38) to be slightly smaller in diameter than it would be in an un-stressed condition. If the difference between Xh and X1 is sufficient to cause deformation of the plug (36) in a vertical direction such that Y is increased to a length greater than Yh, then a pre-stressed vertical compression condition is introduced.

With reference to FIG. 2C, a plug (40) of the type described in FIG. 1 has an outside diameter X and a vertical height Y1. Because Y1 is greater than Yh and X is less than Xh, there exists a pre-stressed condition with respect to the vertical or axial direction when the plug (36) is positioned in the housing (20); but there does not exist a pre- stressed compression condition with respect to the radial direction along the direction of X1. This pre-stressed condition causes the orifice (42) to be slightly smaller in diameter than it would be in an un-stressed condition. If the difference between Yh and Y1 is sufficient to cause deformation of the plug (40) in a horizontal direction such that X is increased to a length greater than Xh, then a pre-stressed horizontal compression condition is introduced.

Referring now to FIG. 2D, because X1 is greater than Xh and Y1 is greater than Yh, there exists a pre-stressed condition with respect to the vertical or axial direction as well as the horizontal or radial when the plug (44) is positioned in the housing (20). This pre-stressed condition causes the orifice (46) to be slightly smaller in diameter than it would be in an un-stressed condition.

In use, the preferred embodiments of the present invention described above operate in the manner described with respect to FIGS. 11A-11C. A trocar housing (48) and attached cannula (50) having a “zero seal” of a known type such as duckbill seal (52) is shown in FIG. 11A. A foam seal plug (54) of the type described above has a central orifice (56) of a diameter of about 4.5 mm (0.177 inches) aligned generally concentrically with the cannula (50). The plug (54) is preferably made of a flexible foam. The plug may be made of an open-cell foam and coated for fluid sealing. Referring to FIG. 11B, a surgical instrument (58) having a handle (60), a shaft (62) of at least slightly greater than 4.5 mm (0.177 inches), and distal end (64) is inserted through the orifice (56) and through the zero seal (52). The zero seal (52) seals insufflation back pressure from traveling up the cannula (50) toward the housing (48) when the seal (52) is in the closed position shown in FIG. 12A. When the shaft (62) is positioned through the zero seal (52), as shown in FIG. 12B, pressure escapes past the shaft (62) and seal (52) through gaps (66), and into the space (68) between the zero seal (52) and the plug (54). The pressure presses against the lower surface (70) of the plug (54) pushing the plug (54) into sealing contact with the underside wall (72) of the top surface (74) of the housing (48). The orifice (56) is stretched to accommodate the instrument shaft (62) in a sealing manner. The vertical height dimension (76) of the plug is of sufficient length such that, in combination with the resiliency and dimensions of the seal plug (54) and dimensions of the cannula (50) and housing (48), horizontal movement of the instrument shaft (62) results in pivoting of the instrument (58) as shown in FIG. 11C. As shown in FIG. 11C, lateral movement of the instrument shaft (62) is limited to the extent that cat-eyeing of the seal plug orifice (56) will not occur. Cat-eyeing is limited since the plug (54) is placed generally in a compressed state by the insertion of the instrument shaft, thereby causing the plug material to decompress and follow the shaft during its movement, in order to fill gaps that would otherwise occur with conventional plug materials.

In an example of the preferred embodiment shown in FIG. 11A, a housing (48) was used having an upper central orifice (76) diameter of 13.5 mm (0.532 inches), a lower central orifice (78) diameter of 18.5 mm (0.730 inches), a cannula inner diameter (80) of 12.3 mm (0.485 inches), a cannula length (82) of 109

mm (4.300 inches), a housing inside diameter (84) of 30.5 mm (1.200 inches), and a housing inside height (86) of 8.9 mm (0.350 inches). A foam plug (54) was used having an orifice diameter (88) of 3.8 mm (0.150 inches), an outside diameter (90) of 3.4 mm (0.1325 inches), a vertical height (92) of 0.200 mm (0.525 inches), and a density of about 12 to 20 pounds per cubic foot. The specific material used for the plug (54) of this example was a flexible foam sold as Pudgee™ and available for sale from Dynamic Systems Inc. of Leicester, N.C. For use with this example, the seal and housing arrangement was tested with pressure applied at its distal end of the ranges encountered in typical laparoscopic procedures and ambient conditions applied at its proximal end. A set of instruments having shafts ranging from 4.5 mm (0.177 inches) to 15 mm (0.590 inches) was used, wherein each instrument was inserted through the orifice (56) and past the zero seal (52), and then moved from side to side within essentially the full range of the central upper orifice (76) in order to move the distal instrument end (64) about its full available movement range, including the tilted condition shown in FIG. 11C. During insertion, manipulation, and removal of the instruments, essentially no measurable amount of pressure escaped past the plug (54) except for the small amount that passed the zero seal (52) but became trapped under the plug (54) until full removal of the instruments from the plug (54).

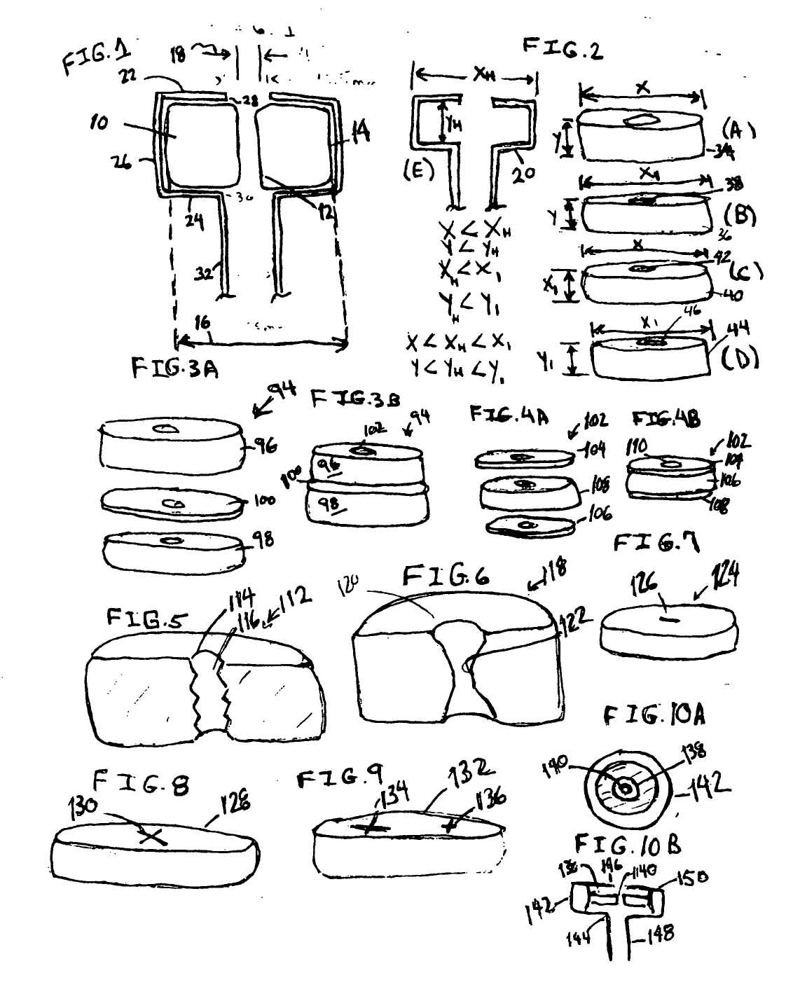

Using other preferred embodiments of the invention, such as those described in FIGS. 3B-3D, where a pre-stressed compression condition exists in which the orifice diameter is made smaller than it would be at rest, the range of deformation of the plug that can be made by a small diameter shaft without causing cat-eyeing is increased because the threshold of where the overall plug condition changes from compression to tension under such conditions is delayed. This increases the ability of the compressed plug material to decompress and follow the shaft during movement and fill the gaps that exist in ordinary seals under like conditions.

Using the same principles described above with respect to the aforementioned embodiments of the present invention, the following alternative embodiments of the present invention may be implemented in a similar manner. In FIGS. 3A-3B, there is shown a foam seal plug assembly (94) comprising a first foam plug (96), a second foam plug (98), and an elastomeric membrane (100) positioned therebetween so that, when assembled, a single central orifice (102) passes therethrough.

In FIGS. 4A-4B, there is shown a foam seal plug assembly (102) comprising a first elastomeric membrane (104), a second elastomeric membrane (106), and a foam plug (108) positioned therebetween so that, when assembled, a single central orifice (110) passes therethrough.

Another embodiment of the present invention, shown in FIG. 5, comprises a foam seal plug (112) having a central orifice (114) comprising multiple internal ridges (116) of smaller diameter to more tightly seal around an instrument shaft.

Yet another embodiment of the present invention, shown in FIG. 6, comprises a foam seal plug (118) having a central orifice (120) comprising an hourglass-shaped profile having a reduced diameter portion (122) for enhanced sealing contact around an instrument shaft.

Referring to FIGS. 7-9, respectively, foam seal plugs (124, 128, 132) of the present invention have one or more slits (126) or cross-slits (130, 134, 136) through the seal plug and adapted to enable passage therethrough of an instrument shaft, while closing completely in the absence of an instrument shaft in order to provide a backup seal to the zero seal or to eliminate entirely the need for any zero seal.

FIGS. 10A-10B illustrate another alternative embodiment of the present invention in which a foam seal plug (138) having a central orifice (140) is positioned within a housing (142) having an inner diameter greater than the outer diameter of the plug (138). The orifice (138) is generally aligned relative to the housing upper and lower openings (144, 146) and cannula (148), while being permitted to float laterally and eccentrically relative thereto utilizing wiper lips (150) to effect a seal between the plug (138) and housing (142).

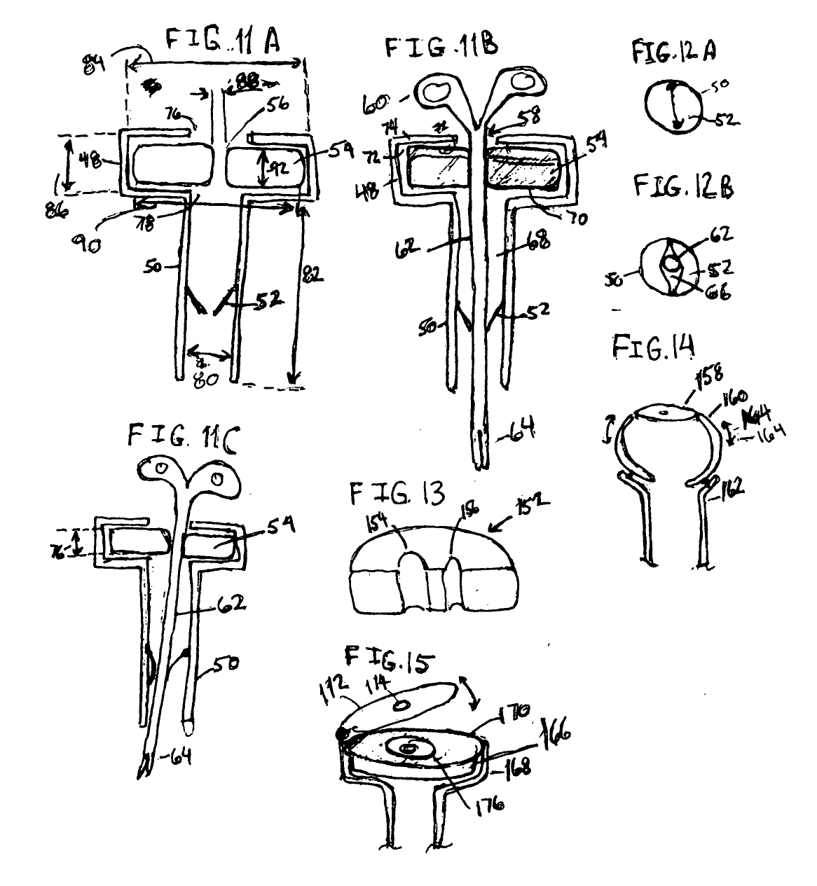

Yet another embodiment of the present invention is described with respect to FIG. 13, wherein a foam seal plug (152) has a plurality of orifices (154, 156) of varying diameters to accommodate various sized instrument diameters.

Another embodiment of the present invention illustrated in FIG. 14 utilizes, in combination, a foam seal plug (158) of any of the aforedescribed types fixedly mounted to a generally spherical head (160) which is movably mounted to a cannula (162) in a manner that facilitates universal pivoting and sliding between the head (160) and cannula (162) along a partially spherical path. This arrangement alleviates cat-eyeing by enabling lateral movement and tilting simultaneously.

Another embodiment of the present invention is illustrated in FIG. 15, and utilizes a foam seal plug (166) contained in housing (168) having a top wall (170) with a first opening (176) and a pivotally attached converter plate (172) having a second opening (174) smaller than the first opening (176). The converter plate (172) can be selectively implemented to restrict lateral movement of an inserted instrument shaft by way of the smaller opening (174) to prevent cat-eyeing.

While the preferred embodiments of the invention have been herein described, it is understood that various modification can be made to the present invention without departing from the scope of the invention.

1. A sealing system for maintaining a pressure barrier between two volumes of different pressure while facilitating the passing of objects therethrough, said system comprising
a foam seal forming a barrier between said two volumes.

2. A system according to claim 1, wherein
said foam seal has at least one passage therethrough for said passing of objects which is in a closed position when said seal is at rest, and which assumes an opened position when said objects are passed through.

3. A system according to claim 1, wherein
said foam seal is comprised of a closed cell foam material.

4. A system according to claim 1, wherein
said foam seal is generally disc-shaped.

5. A system according to claim 1, wherein
said foam seal is generally cylindrical-shaped.

6. A system according to claim 1, wherein
said foam seal is comprised of an opened-cell foam.

7. A system according to claim 1, wherein
said foam seal is comprised of polyurethane.

8. A system according to claim 1, further comprising
a housing structure to hold said foam seal in a position between said volumes in a manner in which said foam seal is compressed.

9. A system according to claim 8, wherein
said seal is compressed in a direction generally parallel to a horizontal plane.

10. A system according to claim 8, wherein
said seal is compressed in a direction generally parallel to a vertical plane.

11. A system according to claim 8, wherein
said seal is compressed in a direction generally parallel to a horizontal plane and generally parallel to a vertical plane.

12. A system according to claim 1, further comprising
a housing structure to hold said foam seal in a position between said volumes, said housing structure having an inside diameter d1;
said foam seal having an outside diameter d2; and
d2 is greater than d1.

13. A system according to claim 1, further comprising
a housing structure to hold said foam seal in a position between said volumes, said housing structure having an interior height h1 representing the distance between the floor and the ceiling of the interior of said housing structure;
said foam seal having a height of h2; and
h2 is greater than h1.

14. A system according to claim 2, wherein
said passage is formed by a slit through said seal.

15. A system according to claim 2, wherein
said passage is formed by a slot-shaped hole through said seal.

16. A system according to claim 2, wherein
said passage is formed by a plurality of slits through said seal that intersect at a common point.

17. A system according to claim 1, further comprising
a plurality of passages formed through said seal.

18. A system according to claim 1, further comprising
a housing structure to hold said foam seal in a position between said volumes, said housing structure having an inside diameter d1;
said foam seal having an outside diameter d2; and
d1 is greater than d2 and said foam seal is movable along a generally horizontal plane.

19. A system according to claim 1, further comprising
a housing structure to hold said foam seal in a position between said volumes, said housing structure having an inside diameter d1;
said foam seal having an outside diameter d2; and
d1 is greater than d2 and said foam seal is anchored against movement along a generally horizontal plane.

20. A system according to claim 1, further comprising
a housing structure to hold said foam seal in a position between said volumes, said housing structure having an interior height hi representing the distance between the floor and the ceiling of the interior of said housing structure;
said foam seal having a height of h2; and
h1 is greater than h2.

21. A system according to claim 1, further comprising
a housing structure to hold said foam seal in a position between said volumes, said housing structure having an inside diameter d1;
said foam seal having an outside diameter d2;
d1 is greater than d2;
said housing structure having an interior height hi representing the distance between the floor and the ceiling of the interior of said housing structure;
said foam seal having a height of h2; and
h1 is greater than h2.

22. A system according to claim 1, further comprising
a housing structure to hold said foam seal in a position between said volumes;
a top opening formed in an upper surface of said housing and positioned generally above said passage; and
a planar adapter adapted to be selectively positioned over said top opening, said adapter having an opening of a diameter different than the diameter of said top opening.

23. A system according to claim 1, further comprising
a housing structure to hold said foam seal in a position between said volumes, said housing comprising a movable component and a stationary component;
said movable component comprising a generally spherical part having a top opening, a bottom opening, and a passage therethrough;
said movable component being mounted to said stationary component; and
said foam seal being mounted across said top opening.

24. A system according to claim 1, wherein
said passage has a maximum horizontal dimension of approximately 4 mm.

25. A system according to claim 24, wherein
said passage is adapted to stretch open to a diameter of approximately 12 mm.

26. A system according to claim 24, wherein
said passage is adapted to stretch open to a diameter of about 15 mm.

27. A system according to claim 1, wherein
said foam seal comprises foam cells of diameters averaging approximately 0.32 mm.

28. A system according to claim 1, wherein
said foam seal comprises closed foam cells of diameters averaging approximately 0.32 mm.

29. A trocar system comprising
a trocar housing having a top, a bottom, a sidewall section, an interior chamber, a top opening in said top and in communication with said interior chamber, and a bottom opening in said bottom and in communication with said interior chamber;
a tube extending downwardly from said housing and being in communication with said interior chamber; and
a foam seal having at least one passage therethrough and being mounted in said interior chamber.

30. A system according to claim 29, further comprising
a second seal positioned in said tube, said second seal having a seal passage therethrough adapted to selectively allow passing of objects through said second seal, and said second seal adapted to seal against pressure exerted upwardly against said second seal.

31. A method of providing an access port in a patient's body, said method comprising
inserting into said patient's body a first end of a tube, said tube being connected at its other to a seal housing;
passing at least one object through a foam seal positioned in said housing and through said tube.

32. A method according to claim 31, further comprising
passing at least one object through a secondary seal positioned in said tube.

33. A method of providing an access port in a patient's body for performing a surgical procedure therethrough, said method comprising
inserting into said patient's body a first end of a tube, said tube being connected at its other to a seal housing;
performing a procedure through a foam seal positioned in said housing and through said tube.

34. A method according to claim 33, further comprising
performing a procedure through a secondary seal positioned in said tube.

35. A system according to claim 1, further comprising
a housing structure to hold said foam seal in a position between said volumes in a manner in which said foam seal is compressed and adapted to remain in compression during passing of objects up to at least 14 mm in diameter through said seal.

36. A sealing system for maintaining a pressure barrier between two volumes of different pressure while facilitating the passing of objects therethrough, said system comprising
a plurality of foam seals forming a barrier between said two volumes.

37. A system according to claim 36, wherein
said plurality of foam seals are stacked relative to one another to form a plurality of generally horizontal layers.

38. A system according to claim 1, further comprising
an elastomeric membrane covering at least one surface of said seal.

39. A system according to claim 36, further comprising
an elastomeric membrane covering at least one surface of one of said seals.

40. A system according to claim 1, further comprising
an elastomeric membrane covering all surfaces of said seal.

41. A system according to claim 36, further comprising
an elastomeric membrane surrounding said plurality of seals.

42. A system according to claim 1, further comprising
a plurality of ribs formed in said passage, said ribs being adapted to flexibly engage an object passing through said passage.

43. A system according to claim 1, wherein
said passage has a varying diameter.

44. A system according to claim 43, wherein
said diameter is smallest in a section of said passage between a top surface and a bottom surface of said seal.
